(19) United States
(12) Patent Application Publication
(10) Pub. No.: US 2008/0275345 A1
(43) Pub. Date: Nov. 6, 2008

(54) Ultrasonic Diagnostic Contrast Imaging at Moderate Mi Levels

(75) Inventors: Matthew Bruce, Seattle, WA (US); Michalakis Averkiou, Kirkland, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/570,602

(22) PCT Filed: Jun. 22, 2005
(86) PCT No.: PCT/IB2005/052055

§ 371 Date: Dec. 14, 2006

(51) Int. Cl. A61B 8/00

(52) U.S. Cl. 600/458

(57) ABSTRACT

A method and device for imaging contrast agents which oscillate nonlinearly in a nondestructive mode at an MI in excess of 0.1 is described. Three transmit pulses are transmitted in each beam direction which are differently modulated. In an illustrated embodiment the transmit pulses are symmetrically differently phase modulated at 0°, 120° and 240°. The echoes received in response to each transmit pulse are stored and combined by a pulse inversion processor. Pulse inversion processing results in separation of the third harmonic to the relative exclusion of the first and second harmonic signal components. Third harmonic images of the contrast image are formed which exhibit a relatively low tissue background.

This invention relates to medical ultrasonic imaging systems and, in particular, to medical diagnostic imaging systems with contrast agents using moderate mechanical index transmit waves.

Ultrasonic imaging of blood flow can be significantly enhanced with the use of ultrasonic contrast agents. The microbubbles of contrast agents can be designed to oscillate nonlinearly or break up when insonified by ultrasound. This oscillation or destruction will cause the echoes returned from the microbubbles to be rich in nonlinear components. The echoes are received and the nonlinear components separated from echoes returned by tissue by filtering or a two-pulse separation technique known as pulse inversion. Images produced with these echoes can sharply segment the blood flow and vasculature containing the contrast agent.

Contrast agents are generally imaged with either high mechanical index (MI) energy or low MI energy. When imaged at a high MI the microbubbles will break or become significantly disrupted, returning strong harmonic echoes. These echoes will show the locations of the broken or disrupted microbubbles in sharp relief against the surrounding tissue. However, several heartbeats are then needed to replenish the imaged area with a fresh flow of new microbubbles before the process can be repeated.

When the microbubbles are images at a low MI they will usually oscillate gently and return harmonic signals and not become disrupted or broken. The returning echoes are not as strong as those returned from high MI pulses but the contrast agent can be continuously imaged in real time as there is no need to replenish the entire image field with a new supply of microbubbles. Contrast agents such as Definity (Bristol-Myers Squibb), Optison (Amersham) and SonoVue (Bracco) have been shown to be effective when imaged at a low MI.

Other contrast agents such as Sonazoid (Amersham) and Biosphere (Accusphere) have been developed to exhibit reduced fragility and thus have an extended lifetime in the presence of ultrasound. It is believed that microbubbles of these contrast agents have a “stiffness” which can resist breakage until higher levels or extended durations of ultrasonic energy are applied. Such contrast agents can be used in lesser infusion doses than more fragile agents and can be useful for imaging within the body for a greater length of time. However the greater stiffness usually requires a higher MI pulse in order to induce the desired nonlinear response from these microbubbles. The higher MI waves will undergo distortion as they pass through tissue and the tissue will return echoes at detectable levels with nonlinear components, the same phenomenon used for tissue harmonic imaging without contrast agents. Thus, the ultrasound system will receive the desired nonlinear echoes from the contrast agent and undesired nonlinear echoes from tissue. At the lower MIs of the more fragile contrast agents, around MI=0.1 or less, the nonlinear tissue response is at a barely detectable level and generally not a problem. But at the more moderate MI's above 0.1 used with the stiffer contrast agents such as levels of 0.3-0.4, the nonlinear contrast agent signals can become contaminated with an unacceptable level of harmonic returns from tissue. Accordingly it is desirable to be able to image contrast agents at moderate MI's but without appreciable contamination by nonlinear signals returned from tissue.

In accordance with the principles of the present invention, a multi-pulse transmit technique is used to image contrast agents at moderate MI's. The pulses are differently modulated so that the nonlinear signals can be separated by pulse inversion processing. In an illustrated embodiment three transmit pulses are phase modulated at 0°, 120°, and 240° and the three resulting echoes combined by pulse inversion processing to separate the nonlinear signals. The modulation of the transmit pulses causes the pulse inversion process to attenuate both the fundamental and second harmonic components, separating a third harmonic component which can be used for imaging with little contamination from tissue.

In the drawings:

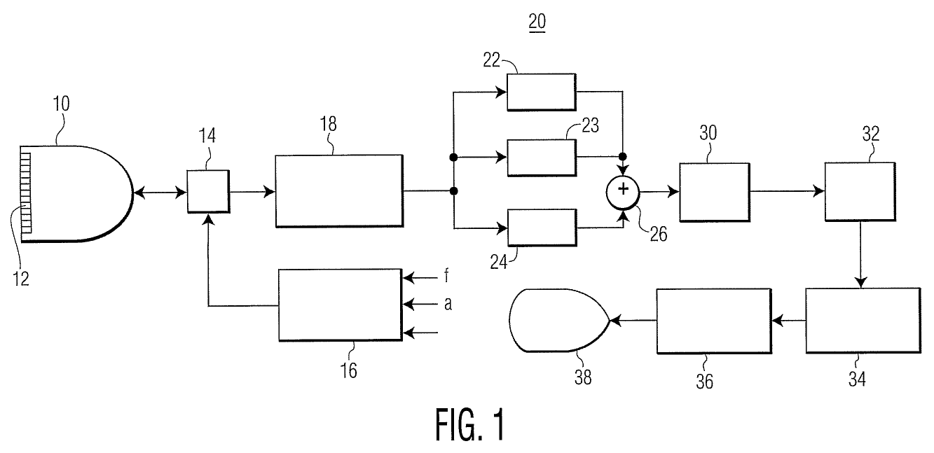

FIG. 1 illustrates in block diagram form an ultrasonic diagnostic imaging system constructed in accordance with the principles of the present invention.

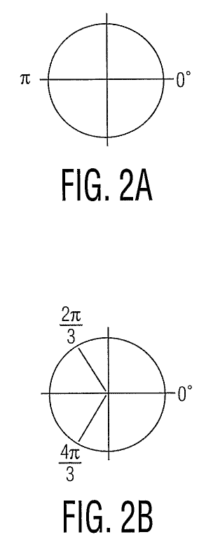

FIGS. 2A-2B illustrate the phases of two-pulse and three-pulse transmit sequences that can be used for pulse inversion harmonic separation.

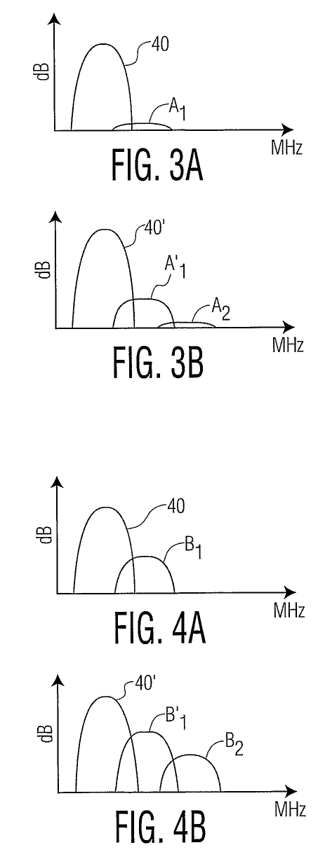

FIGS. 3A through 5B illustrate the result of pulse inversion separation using a three-pulse transmit sequence in accordance with the principles of the present invention.

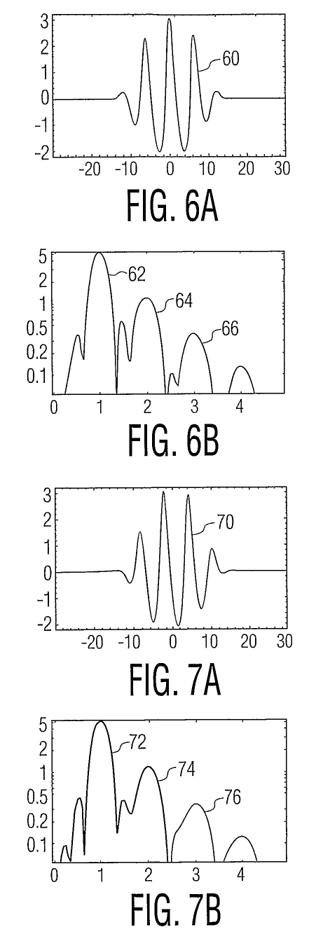

FIGS. 6A through 9B illustrate three differently modulated transmit pulses and the result of pulse inversion processing of their echo signals in accordance with the principles of the present invention.

Referring first to FIG. 1, an ultrasonic diagnostic imaging system constructed in accordance with the principles of the present invention is shown. The ultrasound system of FIG. 1 utilizes a transmitter 16 which transmits multi-pulse sequences for the production of echo signals with nonlinear responses. The transmitter is coupled by a transmit/receive switch 14 to the elements of an array transducer 12 of a scanhead 10. The transmitter is responsive to a number of control parameters which modulate the characteristics of the transmit pulses. The transmitter can control the transmit frequency f of the pulse wave and/or the amplitude a of the pulses. The transmitter can also control the relative phase of the pulse wave. This modulation enables the echoes received in response to the pulses to be combined in order to separate nonlinear echo signal components for imaging.

In FIG. 1, the transducer array 12 receives echoes from the body containing linear and nonlinear signal components which are within the transducer passband. These echo signals are coupled by the switch 14 to a beamformer 18 which appropriately delays echo signals from the different elements then combines them to form a sequence of coherent echo signals along the beam direction from shallow to deeper depths. Preferably the beamformer is a digital beamformer operating on digitized echo signals to produce a sequence of discrete coherent digital echo signals from a near to a far depth of field. The beamformer may be a multiline beamformer which produces two or more sequences of echo signals along multiple spatially distinct receive scanlines in response to a single transmit beam. The beamformed echo signals are coupled to a nonlinear signal separator 20. The separator 20 may be a bandpass filter which passes a frequency band containing nonlinear signals. Preferably the separator is a pulse inversion processor which combines received echo signals to enhance the nonlinear components to the relative exclusion (attenuation) of the linear components. In the illustrated embodiment the separator 20 is a pulse inversion processor which separates the nonlinear signals by combining three received echo signals from the same location. For a three pulse sequence, the scanline echoes received in response to a first transmit pulse in the desired beam direction are stored in a Line1 buffer 22. The scanline echoes received in response to the second transmit pulse in the beam direction are stored in a Line2 buffer 23, and the scanline echoes resulting from a third transmission along the beam direction are stored in a Line3 buffer 24. The echoes from the three buffers are then combined on a spatial basis by a summer 26. Alternatively, the third scanline of echoes may be directly combined with the stored echoes of the first and second scanlines without buffering. As a result of the different modulation of the transmit pulses, the out of phase fundamental (linear) and second harmonic echo components will cancel and the nonlinear third harmonic components, being in phase, will combine to reinforce each other, producing enhanced and isolated nonlinear third harmonic signals. The nonlinear signals may be further filtered by a filter 30 to remove undesired signals such as those resulting from operations such as decimation. The signals are then detected by a detector 32, which may be an amplitude or phase detector. The echo signals are then processed by a signal processor 34 for subsequent grayscale, Doppler or other ultrasound display, then further processed by an image processor 36 for the formation of a two dimensional, three dimensional, spectral, parametric, or other display. The resultant display signals are displayed on a display 38.

In a two-pulse pulse inversion scheme the transmit pulses are modulated in an opposite sense as shown in FIG. 2A. The transmit pulses may be of an opposite polarity or of opposite phases (e.g., 0° and 180°) for full cancellation of the linear signal components. FIG. 2A is a phase drawing illustrating the opposite phases (0° and Π radians) of the transmit pulses of a typical 2-pulse pulse inversion sequence.

Higher order sequences may also be used for pulse inversion such as the three- and five-pulse sequences shown in U.S. Pat. No. 6,186,950 and U.S. patent application Ser. No. 60/527,538. With these sequences three or five echoes received from the same point in the body are combined to separate nonlinear signals by pulse inversion. FIG. 2B illustrates a three-pulse sequence in which the phases of the transmit pulses are uniformly distributed at 120° (2Π/3 radians) increments: 0°, 120° (2Π/3 radians) and 240° (4Π/3 radians). As explained in the paper “A 5-Pulse Sequence for Harmonic and Sub-Harmonic Imaging,” by W. Wilkening et al., in the Abstract Book from the Sixth European Symposium on Ultrasound Contrast Imaging (Jan. 25-26, 2001), a symmetrically phased three-pulse sequence can be used in pulse inversion to cancel the fundamental (linear 1st harmonic) components but will also unfortunately cancel the desired second harmonic. Consequently, Wilkening et al. and other have disdained the use of such sequences for contrast imaging in favor of others that stimulate second harmonic enhancement.

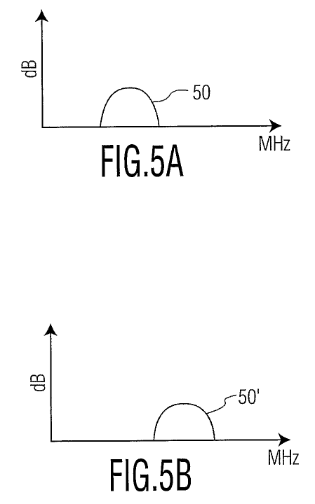

However, the present inventors have discovered that multi-pulse sequences with low second harmonic sensitivity can be advantageously used for imaging with contrast agents at moderate MI's. FIG. 3A illustrates the frequency response from a low MI pulse of MI=0.1, such as would be used with a “soft” contrast agent of microbubbles that oscillate nonlinearly in a nondestructive mode at that MI. The returning echo signal includes a relatively large response 40 at the fundamental transmit frequency. The echo signal also includes a relatively low nonlinear response A₁ at the second harmonic frequency from tissue. The tissue response is relatively low because the transmit pulse intensity of MI=0.1 is relatively low. The echo signal also includes a nonlinear second harmonic component B₁ returned from the microbubbles of the contrast agent as shown in FIG. 4A, which is relatively high. The nonlinear second harmonic components can be separated by pulse inversion by combining the echoes from two differently modulated transmit pulses (e.g., FIG. 2A) which leaves the second harmonic components 50 as illustrated in FIG. 5A. Since the ratio of B₁ to A₁ is relatively large, the second harmonic components can be used to create an image of the contrast agent with little or no tissue harmonic background.

When a “stiffer” contrast agent is imaged which utilizes a higher MI pulse to oscillate nonlinearly such as pulses with MIs in the range of 0.3-0.4, the returning echo component responses are greater. FIG. 3B illustrates the echo components returned from tissue, which include fundamental (1st harmonic) components in the passband 40′ and second harmonic components in the passband A₁′. As compared to the second harmonic response A₁ in FIG. 3A, it is seen that the response A₁′ is greater due to the higher intensity transmit pulse at the higher MI. An echo component A₂ from tissue is also developed at the third harmonic of the transmit frequency. FIG. 4B illustrates the echo components returned from the contrast agent. These include fundamental components in the passband 40′ and nonlinear components B₁′ in the second harmonic band. While the response in the B₁′ band is greater than that in the B₁ band in FIG. 4A due to the higher MI transmit energy, the B₁′ to A₁′ ratio is no longer as favorable as it was at the lower MI. Images formed with these components will exhibit a mixture of contrast agent response and a tissue harmonic background. There is also a third harmonic response B₂ from the microbubble contrast agent and it is seen that the B₂ to A₂ third harmonic ratio is a favorable one. Thus, if a transmit sequence is used in which both the first and second harmonic components can be attenuated or eliminated by pulse inversion processing, the third harmonic band 50′ can be separated containing the third harmonic components with the favorable B₂/A₂ ratio. Images formed with these signals will image the contrast agent with the desirable minimal tissue background.

In accordance with the principles of the present invention, three transmit pulses with relative phase differences of 2Π/3 are used to image a contrast agent at an MI in excess of 0.1. The different phase modulation for the three pulses results in three transmit pulses of the form (up to the third harmonic) of:

p₀(t)=e^{jωt}+e^{j2(ωt)}+e^{j3(ωt)}=e^{jωt}+e^{j2ωt}+e^{j3ωt}

p₁(t)=e^{jωt+2π/3}+e^{j2(ωt+2π/3)}+e^{j3(ωt+2π/3)}=e^{j2π/3}e^{jωt}+e^{j4π/3}e^{j2ωt}+e^{j2π}e^{j3ωt}

p₂(t)+e^{jωt+4π/3}+e^{j2(ωt+4π/3)}+e^{j3(ωt+4π/3)}=e^{j4π/3}e^{jωt}+e^{j8π/3}e^{j2ωt}+e^{j6π}e^{j3ωt}

The echoes received in response to the first transmit pulse p₀(t) are stored in the Line1 buffer 22, the echoes received in response to the second transmit pulse p₁(t) are stored in the Line2 buffer 23, and the echoes received in response to the third transmit pulse p₂(t) are stored in the Line3 buffer 24. The stored echoes are then read out of the three buffers in parallel and combined by the summer 26. The result of this pulse inversion combination of the three echo signals is a signal of the form

p₀(t)+p₀(t)+p₀(t)=0e^{j2ωt}+3e^{j3ωt}

which is seen to contain third harmonic (3ωt) components to the relative exclusion of the first and second harmonic components. Contrast images made with these components will be distinct due to the higher level transmit signals but will be substantially free of a tissue harmonic background.

FIGS. 6A-9B illustrates a series of transmit waveforms for contrast imaging in accordance with the present invention. FIG. 6A shows a first transmit waveform 60 in the time domain. The abscissa of FIG. 6A is time and the ordinate is amplitude. The transmit pulse 60 will produce an echo with a frequency response characteristic as shown in FIG. 6B. In this drawing the abscissa is demarcated in the harmonic order (1^{st} harmonic, 2^{nd} harmonic, 3^{rd} harmonic, etc.) and the ordinate illustrates relative response. As FIG. 6B shows, the fundamental response 62 is the greatest, followed by the second harmonic response 64 and the third harmonic response 66.

FIG. 7A illustrates a second transmit waveform 70 which is modulated with a 2Π/3 phase shift difference relative to the first transmit waveform 60. FIG. 7B shows the response of an echo received in response to waveform 70. The response is seen to contain a first harmonic (fundamental) response 72, a second harmonic response 74 and a third harmonic response 76.

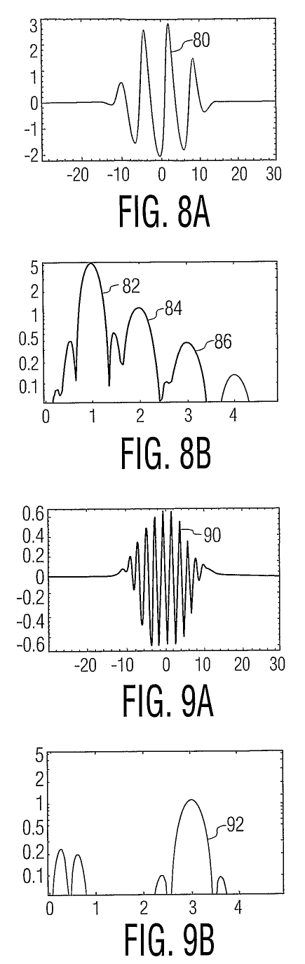

FIG. 8A illustrates a third transmit waveform 80 which is modulated with a 2Π/3 phase shift relative to the first and third transmit waveforms. The three waveforms are thus symmetrically differently phase modulated. Echoes received in response to this transmit waveform have the response shown in FIG. 8B, including a first harmonic response 82, a second harmonic response 84, and a third harmonic response 86.

When the echoes received in response to these three transmit waveforms are combined, the result in the time domain is a waveform 90 as shown in FIG. 9A. The most significant frequency components of the waveform 90 are in the third harmonic band 92 as shown in FIG. 9B. As FIG. 9B shows, there are substantially no components remaining in the first and second harmonic bands, as signals at these frequencies have been canceled in the pulse inversion combination process. Signals in the third harmonic band 92 can be used to make contrast images with little or no contamination from tissue harmonic signal components.

1. A method of separating nonlinear signals returned by an ultrasonic contrast agent comprising:
transmitting a plurality of differently modulated transmit waveforms in a given direction, at least two of which exhibit a phase difference of 2Π/3, at an MI which is in excess of 0.1;
receiving a sequence of echoes in response to each of the transmit waveforms;
combining the received echoes by a pulse inversion process which separates third harmonic components to the relative exclusion of first and second harmonic components; and
forming an ultrasonic image using the separated third harmonic components.

2. The method of claim 1, wherein transmitting further comprises transmitting a plurality of transmit waveforms which are differently phase modulated.

3. The method of claim 2, wherein transmitting further comprises transmitting a plurality of transmit waveforms which are differently phase modulated in a symmetrical manner.

4. The method of claim 3, wherein transmitting further comprises transmitting three transmit waveforms which are differently phase modulated by a phase difference of 2Π/3.

5. The method of claim 1, wherein receiving a sequence of echoes further comprises storing at least the sequences of echoes received in response to first and second transmit waveforms.

6. The method of claim 5, wherein combining the received echoes further comprises combining the stored first and second sequences of echoes with a third received sequence of echoes to separate nonlinear signal components by the pulse inversion method.

7. The method of claim 6, wherein combining the received echoes by the pulse inversion method further comprises separating third harmonic signal components to the relative exclusion of first and second harmonic signal components.

8. The method of claim 7, wherein separating third harmonic signal components further comprises separating third harmonic signal components from a contrast agent to the relative exclusion of coherent fundamental and second harmonic components from tissue.

9. The method of claim 8, wherein forming an ultrasonic image further comprises forming a third harmonic image of a contrast agent which exhibits a relatively low level background tissue image.

10. An ultrasonic diagnostic imaging system which images an ultrasonic contrast agent comprising:
a transducer probe which acts to transmit a plurality of differently modulated transmit waveforms in a given direction, at least two of which exhibit a phase difference of 2Π/3, at an MI in excess of 0.1;
a receiver coupled to the transducer probe which receives a sequence of echo signals in response to each of the transmit waveforms;
a pulse inversion processor coupled to the receiver which separates third harmonic echo signal components to the relative exclusion of first and second harmonic signal components; and
an image processor coupled to the pulse inversion processor which forms third harmonic images of a contrast agent.

11. The ultrasonic diagnostic imaging system of claim 10, wherein the pulse inversion processor further comprises a buffer for storing sequences of echo signals received in response to at least two transmit waveforms.

12. The ultrasonic diagnostic imaging system of claim 11, wherein the pulse inversion processor further comprises first, second, and third buffer memories for storing three sequences of echo signals and a summer coupled to the outputs of the buffer memories for combining signals stored in the buffer memories.

13. The ultrasonic diagnostic imaging system of claim 10, wherein the transducer probe further comprises a transducer probe which transmits three differently phase modulated transmit waveforms in a given direction.

14. The ultrasonic diagnostic imaging system of claim 13, wherein the transducer probe further comprises a transducer probe which transmits three differently phase modulated transmit waveforms in a given direction which are symmetrically phase modulated.

15. The ultrasonic diagnostic imaging system of claim 14, wherein the transducer probe further comprises a transducer probe which transmits three differently phase modulated transmit waveforms in a given direction which are phase modulated at 0°, 120° and 240° relative phase angles.

16. The ultrasonic diagnostic imaging system of claim 14, wherein the transducer probe further comprises a transducer probe which transmits three differently phase modulated transmit waveforms in a given direction which exhibit relative phase differences of 2Π/3.

17. The ultrasonic diagnostic imaging system of claim 10, wherein the image processor further comprises an image processor which forms third harmonic images of a contrast agent with a relatively low background tissue image.
